(19) United States
(12) Patent Application Publication
(10) Pub. No.: US 2020/0163688 A1
(43) Pub. Date: May 28, 2020

(54) AC IMPEDANCE SENSOR SYSTEMS FOR SKIN GRAFT HARVESTING

(71) Applicant: KCI Licensing, Inc., San Antonio, TX (US)

(72) Inventors: Kristine M. KIESWETTER, San Antonio, TX (US); Jeff M. DOLGIN, San Antonio, TX (US)

(21) Appl. No.: 16/630,801

(22) PCT Filed: Jul. 10, 2018
(86) PCT No.: PCT/US2018/041399

§ 371 Date: Jan. 13, 2020

Related U.S. Application Data
(60) Provisional application No. 62/531,712, filed on Jul. 12, 2017.

(51) Int. Cl. A61B 17/322

(52) U.S. Cl. CPC A61B 17/322; A61B 2017/00026; A61B 2017/3225

(57) ABSTRACT

Skin graft harvesting systems and methods based on impedance monitoring are disclosed that utilize impedance sensors to automate the harvesting of skin grafts or assist a user in deciding when the skin graft is ready to be harvested. Such systems and methods can reduce the burden of visual observation and ensure greater reliability and consistency of the grafts. The systems, methods, and devices disclosed herein are particularly useful with harvesters that rely upon suction and/or heating to raise a plurality of small or “micro” blisters simultaneously.

### RELATED APPLICATIONS

This Application claims the benefit of and priority to U.S. Provisional Application No. 62/531,712, filed on Jul. 12, 2107, the entire teachings of which is incorporated by reference herein.

### FIELD

The present disclosure generally relates to devices and methods for generating and harvesting skin grafts.

### BACKGROUND

Skin is the largest organ of the human body, representing approximately 16% of a person's total body weight. Because it interfaces with the environment, skin has an important function in body defense, acting as an anatomical barrier from pathogens and other environmental substances. Skin also provides a semi-permeable barrier that prevents excessive fluid loss while ensuring that essential nutrients are not washed out of the body. Other functions of skin include insulation, temperature regulation, and sensation. Skin tissue may be subject to many forms of damage, including burns, trauma, disease, and depigmentation (e.g., vitiligo).

Skin grafts are often used to repair such skin damage. Skin grafting is a surgical procedure in which a section of skin is removed from one area of a person's body (autograft), removed from another human source (allograft), or removed from another animal (xenograft), and transplanted to a recipient site of a patient, such as a wound site. Typically it is preferable to use an autograft instead of an allograft or a xenograft to reduce complications, such as graft failure and rejection of the skin graft.

A problem encountered when using an autograft is that skin is taken from another area of a person's body to produce the graft, resulting in trauma and wound generation at the donor site. Generally, the size of the graft matches the size of the recipient site, and thus a large recipient site requires removal of a large section of skin from a donor site, leading to increased pain and discomfort and longer healing time. Additionally, as the size of the section of skin removed from the donor site increases, so does the possibility of infection.

Techniques have been developed for harvesting a large number of smaller grafts, e.g., so-called micrografts, to reduce the trauma at the donor site. By removing only a fraction of the skin at a donor site and leaving regions of healthy skin surrounding the excised regions, a large amount of skin for transplantation can be obtained with less discomfort. Micrograft harvesting can also reduce the healing time and risk of infection at the donor site.

One current mode of obtaining epithelial grafts involves separating epidermis micrographs from the dermis by securing a rigid plate with circular holes in it over the donor site. This plate is then coupled to a device that administers heat and/or vacuum to the donor site. The separation of the dermal-epidermal junction occurs as blisters form within the pre-cut holes of the donor site template. As the separation occurs the blisters evolve from an opaque, matte appearance to translucent, shiny and pearlescent and can be visualized through a port or window in the coupled device. The time frame for this separation, however, varies considerably based upon the individual's age and donor site location. Furthermore, the ability to determine if complete separation has occurred requires expertise in order to avoid harvesting the grafts prematurely.

At present, micrografts are deemed to be ready for harvest based upon visual assessment. By looking into the window of the harvester head, the micrografts can be viewed to determine if they have sufficiently formed, that is that the epidermis has separated from the underlying dermis so that when the harvester blades are activated the epidermis grafts can be harvested from the donor site.

Commonly-owned U.S. Published Patent Application No. 2015/0182242 discloses certain sensor systems for skin graft harvesting. The systems disclosed in this patent application are primarily optical (line of sight) or electrical (contact) in nature. Although these systems may provide one solution to problem, there may be a need, nonetheless, for additional or perhaps more sensitive or simpler sensor techniques for monitoring micrograft blister formation.

### SUMMARY

Skin graft harvesting systems and methods are disclosed that utilize alternating current (AC) impedance sensors to assist a user in deciding when the skin graft is ready to be harvested. Such systems and methods can reduce the burden of visual observation and ensure greater reliability and consistency of the grafts. The embodiments disclosed herein is particularly useful with harvesters that rely upon suction and/or heating to raise a plurality of small or “micro” blisters simultaneously.

In some embodiments, the sensor systems can include an AC electrical current source connected to one or more interrogating electrodes and one or more receiver electrodes connected to detector circuitry to monitor changes in the AC impedance. The AC electrical current source produces a signal that will be received by the detector circuitry via the receiver electrode(s).

In some embodiments, a single interrogating electrode and a single receiver electrode can suffice to monitor a plurality of forming blisters. A change in the detected signal can provide an indication that all of the blisters (or at least most of the blisters) are ready for harvesting.

In certain embodiments, the system can include a plurality of interrogating electrodes and/or a plurality of receiver electrodes arranged such that a change in the detected current signal can indicate when a skin blister reaches a point suitable for harvesting. For example, a single interrogating electrode can be paired with multiple receiving electrodes. Utilizing a single signal transmitter paired with multiple receivers can reduce complexity and cost by reducing the number of components in the system.

The interrogation signal can be an alternating current signal, e.g., a sinewave at a frequency in the range of about 10 Hz to about 10 MHz, or some instances, ranging from about 100 Hz to about 2.5 MHz. In some instances, the frequency can be a low or medium radio frequency, e.g., about 30 kHz to about 3 MHz. In some instances it can be advantageous to employ a variable (chirped) frequency that is swept over a preselected range. For example, the frequency can be swept from 100 Hz (or 1 kHz or 10 kHz) to 1 MHz during the course of an interrogation. Sweeping can be performed in a continuous or discrete manner. For example, in discrete step sweeping, measurements can be taken at 10 Hz, 100 Hz, 1 kHz, etc. Any other series of frequencies can be substituted for this example series—and the sequence can be an “up-chirp,” a “down-chirp,” or a hybrid combination of frequencies. Due to the dynamic nature of blister formation, it can be advantageous to take repeated sensor readings before an alert is given that the site is ready for harvesting. For example, pulses at various frequencies can be sent out via the interrogating electrode at intervals, e.g., predetermined intervals such as every second or every 5 seconds.

When the detector senses a signal change (or lack of change for a period of time) it can be assumed that the blister has achieved a sufficient height ready for harvesting. For example, in a single receiving electrode embodiment, the impedance measurements can initially continuously vary, and reach a steady state value as most or all of the blisters reach maturity. If a plurality of receiving electrodes are deployed, e.g., one for each blister, the detected circuitry can track each blister formation individually. In multiple blister (microblister) systems with a plurality of sensor circuits, it can also be desirable to set a threshold, e.g., when a pre-assigned number of blisters are in a “ready” state, before giving an alert to the user that the site is ready for harvesting.

When the conditions have been met or a determination is otherwise made by the system that the skin is ready for harvesting, the device can instruct the user to do so. Alternatively, the system can be automatic, e.g., actuating the harvester to cleave the blisters as soon as certain conditions are met.

In another aspect, methods for preparing a skin graft can include applying a blister cutting device to a donor site on a subject's skin; applying a negative pressure within the device to thereby raise one or more blisters at the donor site; and monitoring the formation of the blister with an AC impedance sensor. The method can further include transmitting information from the sensor to alert a user when at least one blister has reached a state suitable for harvesting, e.g., by transmitting information from the impedance sensor to a controller. Upon receipt of the information from the sensor, the controller can reduce or terminate the negative pressure, alert the user, and/or activate the cutter device (blister cutting device) to cleave the blister.

In some embodiments, monitoring the formation of the blister within the device can further include deploying a sensor comprising at least one interrogating and receiving electrode pair within the device, emitting a signal from an AC source via the interrogating electrode, and receiving the signal with the receiver electrode. For example, the emitter can emit a radio frequency signal (or a chirped radio frequency signal) and the receiver electrode and detector circuitry can be capable of detecting such radiation or acoustic wave. A change (or lack of change) in the signal reception can indicate the presence of a skin blister.

In another aspect, the method can employ a cutting device that includes at least one fixed plate and at least one movable cutter plate, each plate having a plurality of holes through which suction blisters can be raised when the holes of the fixed and movable plates are aligned. The method can include cleaving the blisters, wherein cleaving the blisters comprises moving the movable cutter plate to disrupt alignment of the holes and separate the blisters from remaining skin at the donor site. The method can further include delivering a warm hypotonic fluid to a chamber within the device such that skin exposed to the chamber via the plate holes can assimilate fluid before and/or after applying negative pressure to the chamber to pull skin into the chamber through the holes and thereby raise a plurality of blisters.

More specifically, the methods for preparing a skin graft can be practiced with a device comprising a device body, a sealing head member, at least one fixed plate and at least one movable cutter plate, each plate comprising a plurality of holes and wherein as assembled the holes in the plates are aligned within the body. The method can include: connecting the device to a donor site on a subject's skin; joining the sealing head member and body together to define a sealed chamber; applying negative pressure and/or temperature to the chamber to pull skin into the chamber through the holes and thereby raise a plurality of blisters; monitoring the formation of blisters with one or more sensors; detecting when the blisters are in a condition suitable for harvesting (e.g., based on a detected change or lack of change in an interrogation signal); unsealing the chamber; applying an adhesive substrate to the exposed blisters within the chamber; actuating the movable cutter plate to disrupt the alignment of the holes and to cut the blisters; and removing the substrate together with the cleaved skin blisters.

In another aspect, devices are disclosed for obtaining a skin graft. The devices can include a body that is disposable on a patient's skin; a head adapted for coupling to a cutting body, the head further comprising a sealing surface to engage with a mating surface on the cutting body such that, when the head is engaged with the body on a patient's skin, a sealed chamber is formed over a target region of skin; and a negative pressure conduit also connected to the chamber and adapted for applying negative pressure and/or controlled temperature within the chamber to raise at least one skin blister within the chamber; an impedance sensor for monitoring the formation of the blister; and a cutter assembly within the body for cleaving the blister after formation.

In some embodiments, the sensor is configured to alert the user when the blister has reached a state suitable for harvesting. Alternatively, the device can also include a controller for receiving information from the detector circuitry. The controller can be configured to perform at least one of a) reduce or terminate the negative pressure based on the information received from the sensor and b) activate the cutter assembly and cleave the blister based on information from the sensor.

In certain embodiments, the sensor can include at least one interrogating-receiving electrode pair within the device such that the interrogating electrode is configured to emit a signal and the receiving electrode is configured to receive the signal, and wherein a change in signal indicates the presence of a skin blister. A change in signal, as used herein, encompasses detection of no further changes in impedance or an asymptotic approach to a steady state condition. The sensor is situated within the device such that it can be deployed in close proximity to a growing blister, and configured to sense the presence, e.g., the size and/or height of the blister (or simply contact) by detecting changes in AC impedance.

In certain embodiments, a two part device for harvesting of skin microblisters is disclosed. The two parts are a harvester body is adapted for attachment to a target region of skin and a harvester head, which delivers negative pressure and/or heat to at least portions of the skin engaged by the harvester body.

More specifically, the head is adapted for coupling to a cutting body (‘harvester”) that is disposable on a patient's skin and further adapted for coupling to a vacuum source, the head can further provide a sealing surface to engage with a mating surface on the cutting body such that, when the head is engaged with the cutting body on a patient's skin, an evacuated chamber is formed over a target region of skin; and, preferably, the sensor system is disposed within the chamber to monitor blisters as they are formed.

Optionally, in addition to defining at least a portion of a negative pressure chamber, the head can further include a heating element such as a resistive electrical heating element, or an assembly for infusing a heated fluid. In such systems, in addition to the blister-monitoring sensors, the head or harvester can also include at least one temperature measuring element, such as a thermistor, for measuring the temperature of the skin or evacuated chamber.

The harvester body is configured for placement on a target region of a patient's skin and further adapted to form a sealing engagement with a head and define the chamber for application of negative pressure. In one embodiment, the harvester body can further include at least one alignment plate having a plurality of holes through which skin blisters can be raised in the presence of negative pressure; and a cutting plate having at least one cutting surface for cleaving skin blisters after they are formed within the chamber.

In another preferred embodiment, the harvester can include a top alignment plate and a bottom alignment plate and the cutting plate is disposed therebetween. The top and bottom alignment plates can be joined together by a plurality of vertical posts that pass through slots in the cutting plate to maintain the fixed position of the top and bottom plates relative to each other while permitting movement of cutting plate. The top plate, bottom plate, and cutting plate can each have a plurality of holes that are adapted to be concentrically aligned to facilitate blister formation. In certain embodiments, the holes of the top plate can be larger than the holes of the bottom plate.

The cutting plate can include a plurality of holes suitable for concentric alignment with holes in the alignment plate in a first position to facilitate blister formation and a plurality of cutting surfaces suitable for cleaving blisters in a second position. The cutting plate can be joined to either the top plate or the bottom plate by one or more frangible connectors, such that the frangible connection(s) can be broken by an actuator applying a force to the cutting plate. Once the frangible connection is overcome, the applied force moves the cutting plate to occlude the holes and thereby sever the formed blisters from the subject's skin. The harvester can further include an actuator for moving the cutting plate from the first position to the second position and the actuator can be configured to also at least partially retract the cutting plate following blister cleavage.

The sensor systems disclosed herein can be disposed in proximity to the cutter assembly (e.g., adjacent to, or incorporated into, the top plate or bottom plate to monitor blister formation). Signals from the sensor element(s) can be transmitted to a controller, e.g., a microprocessor or programmed logic unit, which can be disposed in the head, the harvester or in a remote console. Alternatively, the sensor elements can include a transmitter that wirelessly transmits information regarding blister formation to a remote terminal or controller.

When sensor detects an endpoint, the skin is ready for harvesting. The controller can shut off the blister forming elements of the harvester or alert the user that the blisters can be harvested.

The potential advantages of the embodiments disclosed herein in the context of suction blister devices can include (a) reducing the burden on the caregiver in terms of monitoring and constant visual checking, (b) providing more accurate determinations of when the blisters are in condition for harvesting, (c) reducing the time for harvesting since the system will alert the care giver as soon as the site is ready, and/or (d) reducing discomfort for patient as system is currently monitored manually by removing the top of the device if the user cannot judge the state of blisters through the viewing window.

These and other aspects of the methods, systems and devices disclosed herein are described in the figures, description and claims that follow. While several improved design features have been individually described, such features are not mutually exclusive of each other. Any combination of design features disclosed herein can be used integrated into the methods, devices, and systems disclosed herein. These design features and other aspects of the methods, devices and systems disclosed herein are described in the figures, description and claims that follow.

### BRIEF DESCRIPTION OF THE DRAWINGS

FIG. 1A is a schematic perspective view of a skin blister harvesting device according to some embodiments disclosed herein;

FIG. 1B is a schematic perspective view of a base portion of the skin blister harvesting device of FIG. 1A;

FIG. 2 is a schematic illustration of a sensor system employing impedance measurements to monitor blister formation;

FIG. 3 is a bottom view of a harvester device with a sensor system to some embodiments disclosed herein, in which an interrogating electrode is disposed on the skin-contacting surface of the harvester device;

FIG. 4 is a top view of a harvester device with a sensor system according to some embodiments disclosed herein, in which a receiving electrode is disposed above the cutting assembly of the harvester device;

FIG. 5 is a schematic illustration of an impedance sensing harvester connected to an impedance meter according to some embodiments disclosed herein;

FIG. 6 is a schematic exploded view of an embodiment of a harvester according to some embodiments disclosed herein showing components of the cutting assembly and the interrogating and receiving electrodes;

FIG. 7 is a schematic side view illustration of a sensor system according to FIGS. 3-4 at an initial stage in which the harvester has been placed on a donor region of a subject's skin prior to blister formation;

FIG. 8 is a schematic side view illustration of a sensor system of FIG. 7 at an intermediate stage where blister begins to form;

FIG. 9 is a schematic side view illustration of a sensor system of FIG. 7 at an intermediate stage where blister is fully formed;

FIG. 10 is a schematic side view illustration of a sensor system of FIG. 7 illustrating application of a transfer substrate to the formed blisters and their cleavage from the skin;

FIG. 11 is a schematic side view illustration of a sensor system of FIG. 7 illustrated removal of the transfer substrate with cleaved blisters attached thereto;

FIG. 12 is a schematic top view of an alternative embodiment for either the interrogatory or receiving electrode or both.

FIG. 13A is a schematic illustration of a controller display showing a user interface for selecting a desired blister height;

FIG. 13B is a schematic illustration of a controller display showing a user interface for selecting a desired blister fill factor; and

FIG. 13C is a schematic illustration of a controller display showing a user interface for showing progress in successful blister formation.

### DETAILED DESCRIPTION

The present disclosure generally relates to sensor systems for use in devices that can raise a blister (e.g., a suction blister) and cut the raised blister, e.g., a blister raising device integrated with a cutting member. Such devices are useful for harvesting skin grafts. In particular the devices and systems are adapted to infuse a fluid into skin at a donor site to enhance blister formation.

In certain embodiments, the devices according to some embodiments disclosed herein can include a head portion that can be removably coupled to a harvester body that can be positioned at the donor site of a subject's skin. The head portion and the body portion can define a sealed chamber therebetween so that negative pressure can be applied to skin. The sealed chamber can also facilitate fluid instillation and removal, e.g., a heated and/or hypotonic fluid, so that such a fluid can be applied to skin prior to the application of negative pressure. Although shown and described as part of the head portion, it should be clear that the coupler or conduit for fluid delivery and evacuation of the chamber can be part of either the head portion or the body portion and that the fluid and negative pressure can be applied separately via multiple couplings or via a single conduit as illustrated.

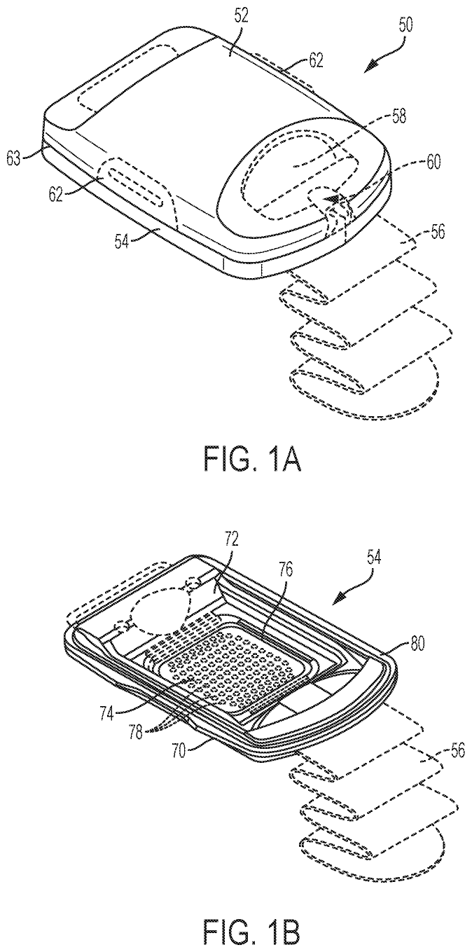

FIG. 1A is a schematic view of a skin graft harvester 50 for use in accordance with various aspects of the present teachings. In this illustrative embodiment, the harvester 50 can include a detachable head portion 52 and harvester body 54. The harvester body 54 is adapted for placement on a patient's skin at a donor site where skin grafts are to be obtained, e.g., on the inner thigh, and secured in place, for example, with strap 56 (shown in phantom). The head 52 can further include a heater (not shown) powered via a coupler 60 adapted to couple with a power source in a base unit (not shown). The head 52 further includes a seal 63, which permits a reduced pressure chamber to be formed when the head 52 and body 54 are joined together and the harvester 50 is coupled to a vacuum pump or other source of reduced pressure, e.g., via coupler 60 connecting the harvester 50 to its base unit. The head 52 can further include one or more windows 58 for observation of skin blisters being formed within the chamber by application of reduced pressure, heat or both. Once the blisters have been formed, the head 52 can be removed, e.g., by deactivating the source of reduced pressure and by actuation of release levers 62, which break the seal 63 and allow the head 52 to be lifted off the harvester body 54.

Additional details on harvesters useful in connection with the embodiments disclosed herein can be found in U.S. patent application Ser. No. 13/839,518 filed Mar. 15, 2013; U.S. patent application Ser. No. 13/346,329 filed Jan. 9, 2012; U.S. patent application Ser. No. 13/436,318 also filed Jan. 9, 2012; U.S. patent application Ser. No. 13/014,737 filed Jan. 27, 2011; U.S. patent application Ser. No. 12/851,656 filed Aug. 6, 2010; U.S. patent application Ser. No. 12/851,621 filed Aug. 6, 2010; U.S. patent application Ser. No. 12/851,703 filed Aug. 6, 2010; and U.S. patent application Ser. No. 12/851,682 filed Aug. 6, 2010. The content of each of the above-referenced related applications is herein incorporated by reference in its entirety.

FIG. 1B is a schematic view of the skin graft harvester 50 of FIG. 1A with the head 52 removed and the cutting assembly 74 exposed. The harvester body 54 can include a base portion 70, a sled 72, and actuator handle 80. The cutting assembly 74 can include a plurality of plates with initially aligned holes through which skin blisters are drawn by heat and/or application of suction when the head 52 is joined to the harvester body 54 and activated. Once the blisters are formed, they can be cleaved by the cutting assembly 74. For example, below the top plate depicted in FIG. 8, one or more additional plates, e.g., a cutter plate and a bottom plate can be deployed with aligned holes 78. By actuation of (e.g., pulling up on) handle 80, the sled 72 is caused to move horizontally such that one of the plates below the top plate, e.g., the “cutter plate” (not shown) also moves (because of its linkage to the sled 72), thereby occluding the alignment of holes 78 and cleaving the raised blisters from the donor's skin.

As explained in more detail below, the sensor systems can be incorporated into the harvester body 54. For example, one or more interrogating electrodes can be incorporated into the bottom 70 of the harvester body and configured to contact the subject's skin in the donor region. One or more receiving electrodes can be incorporated in to the harvester body bed 76, e.g. on top of the cutter assembly 74, as described in more detail below. It may also be desirable to separate either the receiving electrodes or the interrogating electrodes—or both—from the cutter assembly 74 by an electrically insulating material to avoid spurious signals, especially if the cutter assembly is composed of conductive metallic components.

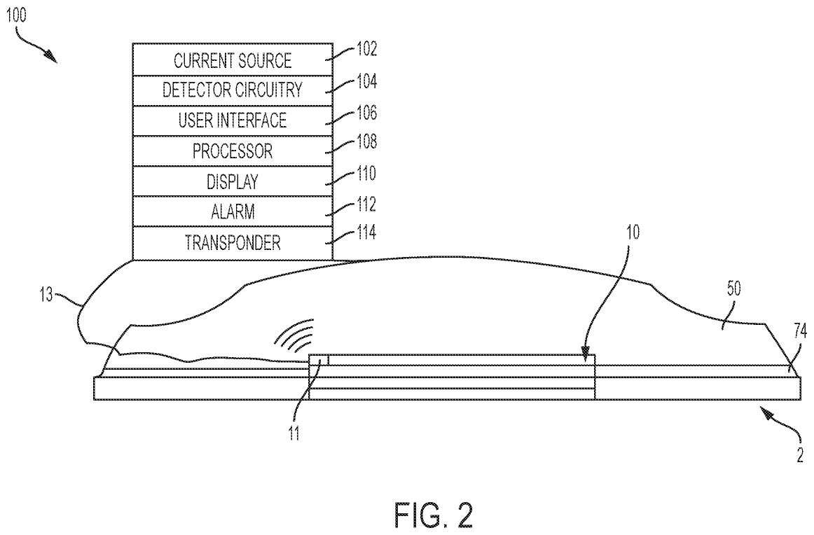

FIG. 2 is a schematic illustration of harvester device 50 having a cutter assembly 74 in contact with a donor site of a patient's skin 2 and equipped with an impedance sensor 10 to reduce burden and variability in deciding when a skin graft is ready to be harvested. The impedance sensor 10 is preferably an AC impedance sensor; however, in some instances a DC sensor can be substituted. The sensor is connected (e.g., by a wiring conduit 13 or wireless path via transceiver 11) to a controller 100 which can be part of the harvester or situated remotely (e.g., as part of the console that provides a source of negative pressure and/or current to heater elements (not shown) within the harvester). The controller 100 can be a dedicated device or a software application on general purpose computer, laptop, tablet or smart phone type device. The wireless connection can operate via a Bluetooth or other communication protocol. In certain embodiments, the controller will include a current source, e.g., an AC current source, detector circuitry, a data processor and a display.

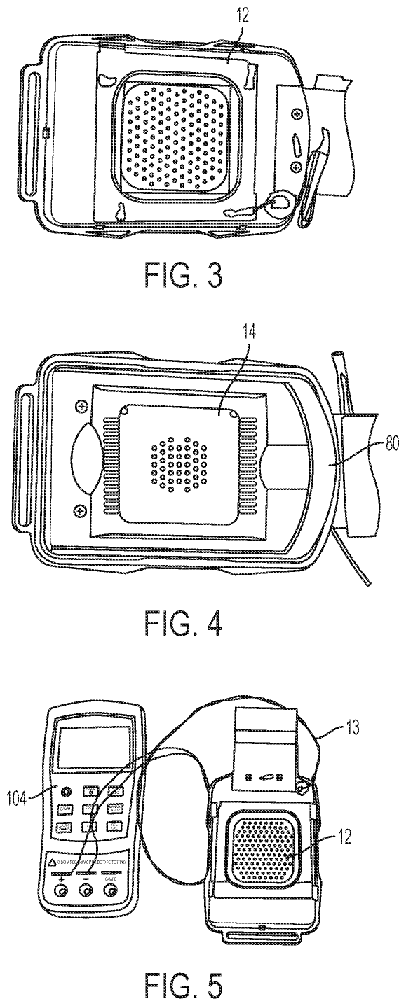

FIG. 3 is a bottom view of a harvester device with a sensor system according to some embodiments, in which an interrogating electrode is disposed on the skin-contacting surface of the harvester device. FIG. 4 is a top view of a harvester device with a sensor system according to one embodiment, in which a receiving electrode is disposed above the cutting assembly of the harvester device. FIG. 5 is a schematic illustration of an impedance-sensing harvester connected to detector circuitry (an impedance meter) according to some embodiments disclosed herein.

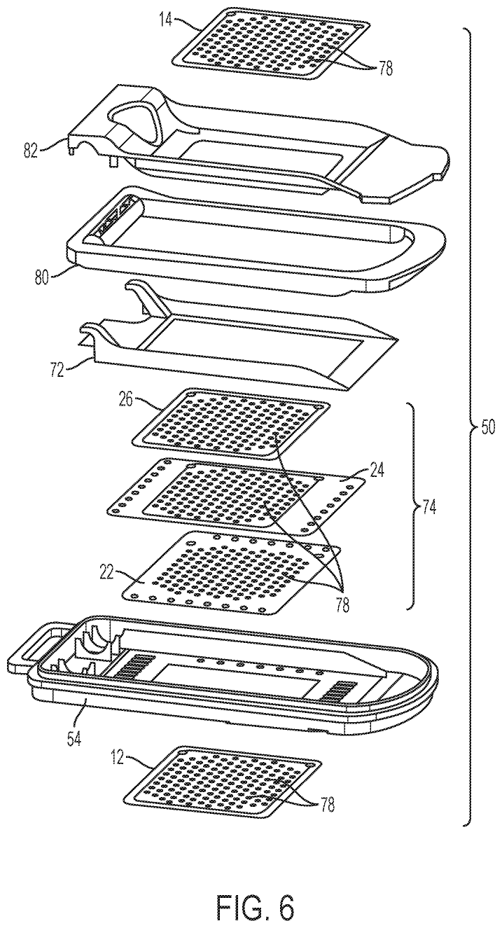

FIG. 6 is a schematic exploded view of an embodiment of a harvester according to some embodiments disclosed herein showing components of the cutting assembly and the interrogating and receiving electrodes. Harvester 50 includes a bottom element 54 with a strap coupler (not shown) e.g., for joining a hook and fastener-type strap to the harvester to facilitate attachment of the harvester 50 to a patient's skin, e.g., by wrapping the device around a patient's leg for harvesting skin from the inner thigh. The harvester 54 also includes a cutter assembly 74 with a bottom plate 22, a top plate 26 and a middle (cutter) plate 24 configurable to initially provide concentrically (coaxially) aligned holes 78 through which blisters can be raised. The harvester 50 further includes a cutter drive sled 72, handle actuator 80 and a top element 82.

In the illustrated embodiment, a single interrogating electrode 12 is disposed on the skin-contacting surface at the bottom of harvester body 54. The interrogating electrode includes a plurality of holes aligned with the holes 78 of the cutter assembly 74. Another unitary receiving electrode 14 with a likewise aligned plurality of holes is disposed above the cutter assembly 74 in the bed of the harvester body 54. One or both electrodes 12, 14 can be separated from the cutter assembly 74 by an insulating layer (not shown). All of the aforementioned holes are coaxially aligned so that blisters can be induced to form in the holes and ultimately reach (or protrude) from the holes of the receiving electrode 14. The holes can be the same size or different. For example, in certain embodiments, it may be desirable for the holes in the receiving electrode to be larger. Alternatively, the holes in the interrogating electrode can advantageously be larger in some applications.

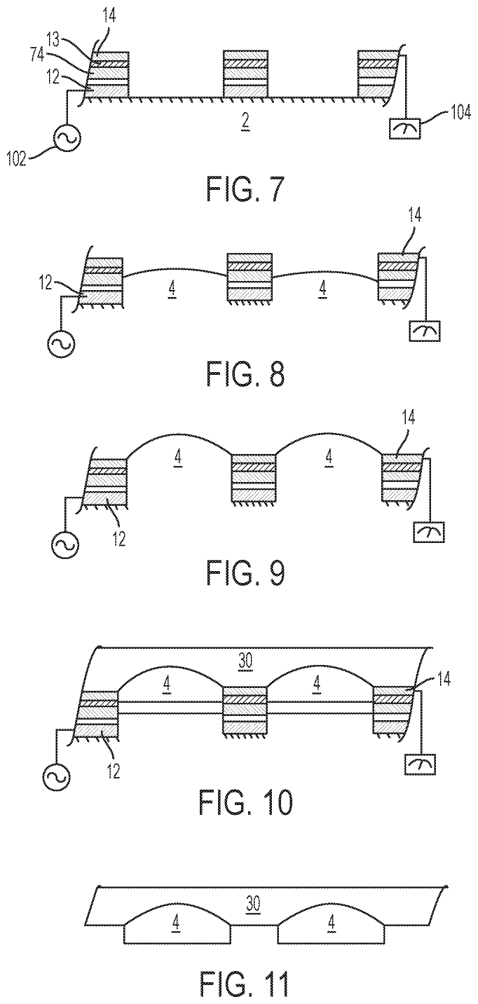

FIG. 7 is a schematic side view illustration of a sensor system according to the invention at an initial stage in which the harvester has been placed on a donor region of a subject's skin prior to blister formation. At this stage the skin is parallel to the bottom of the harvester. FIG. 8 is a schematic side view illustration of a sensor system of FIG. 7 at an intermediate stage where blisters 4 begin to form. The skin is drawn through into the holes of the interrogating electrode 12 and cutter assembly 74. FIG. 9 is a schematic side view illustration of a sensor system of FIG. 7 at an stage where blister 4 is fully formed and has been drawn through all of the holes (i.e., in the interrogating electrode 12, cutter assembly 74, insulating layer 13 and receiving electrode 14. At this point, in some implementations, the impedance measurements can reach a steady state level—indicating the micrografts are ready for harvest and an alert can be sent to the clinician. FIG. 10 is a schematic side view illustration of a sensor system of FIG. 7 illustrating application of a transfer substrate 30 to the fully-formed blisters and their cleavage from the skin. FIG. 11 is a schematic side view illustration of the removal of the transfer substrate with cleaved blisters 4 attached thereto.

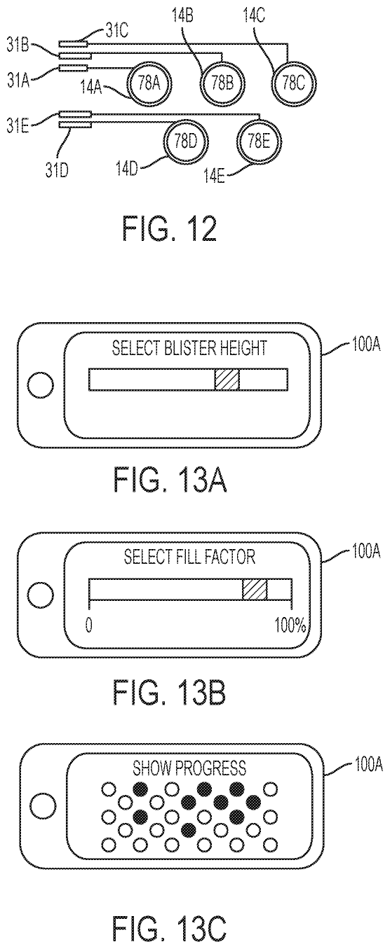

FIG. 12 illustrates an alternative embodiment in which a unitary receiving electrode (and/or a unitary interrogating electrode) is replaced by individual electrodes 14A-14E surrounding each hole 78A-78E each having its own wiring 31A-31E, respectively, to ensure that every blister is individually monitored during formation. In this embodiment, when a threshold number of impedance measurements reach a steady state level the clinician can be alerted that micrografts are ready for harvest.

Accordingly, based on impedance measurements the device can instruct the user that micrografts are ready for harvesting. Depending on the complexity of the device, the system may also indicate the next steps in the process.

In some embodiments, the device/sensor can be configured to allow the user to define the blister height before initiating graft formation. This can be done, for example, by setting a timing delay. For example, as shown in FIG. 13A, the controller can include a handheld user interface 100A, e.g., a smartphone app, that can communicate with the sensor system 100 to select a desired height.

The device/sensor can also be configured to allow the user to define the minimal number of successfully formed grafts that need to be formed before alerting the clinician that the grafts are ready (50%, 70%, 100%). In addition, the device can count the number of grafts formed and either display as a total, a percentage of the total, or in a light grid pattern (one light for each graft, changing from red to green when formed). This allows the user to decide if they have enough grafts and in the pattern/orientation suitable for them. For example, as shown in FIG. 13B, the handheld user interface 100A can communicate with the sensor system to select a desired fill factor to be obtained or, as shown in FIG. 13C, the handheld user interface 100A can monitor and display the progress of blister formation so that the user can determine when to initiate graft formation.

The term “in proximity” encompasses situations wherein objects are close to each other as well situations where objects are in contact with each other. Closeness is not absolute quantity but rather denotes a distance wherein an object, e.g., a sensor, can perform its intended function.

References and citations to other documents, such as patents, patent applications, patent publications, journals, books, papers, web contents, have been made throughout this disclosure. All such documents are hereby incorporated herein by reference in their entirety for all purposes.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

1. A method for preparing a skin graft, the method comprising:
applying a blister cutting device to a donor site on a subject's skin;
applying a negative pressure within the blister cutting device to thereby raise one or more blisters at the donor site; and
monitoring the formation of the blister with an alternating current (AC) impedance sensor.

2. The method of claim 1, further comprising applying heat to the donor site to assist in blister formation.

3. The method of claim 1, further comprising transmitting information from the AC impedance sensor to alert a user when at least one blister has reached a state suitable for harvesting.

4. The method of claim 1, further comprising transmitting information from the AC impedance sensor to a controller configured to initiate a reduction or a termination of the negative pressure based upon information from the AC impedance sensor.

5. The method of claim 1, further comprising transmitting information from the AC impedance sensor to a controller configured to activate the blister cutting device to cleave at least one formed blister.

6. The method of claim 1, further comprising monitoring the formation of the blister within the blister cutting device by deploying a sensor, comprising at least one interrogating electrode and at least one receiving electrode, within the blister cutting device, whereby the at least one sensor is configured to emit a signal from the at least one interrogating electrode and receive a return signal with the at least one receiving electrode.

7. The method of claim 6, wherein the at least one interrogating electrode is connected to a source of an alternating current having at least one frequency in a range from about 10 Hz to about 10 MHz, and the at least one receiving electrode is connected to detector circuitry capable of detecting said alternating current, whereby a change in impedance indicates presence of at least one skin blister.

8. The method of claim 7, wherein the alternating current comprises at least one frequency in a range from about 100 Hz to about 2.5 MHz.

9. The method of claim 7, wherein the frequency is swept over a plurality of frequencies.

10. The method of claim 6, wherein the receiving electrode is disposed in close proximity to a growing blister and configured to sense presence of a blister.

11. The method of claim 1, further comprising applying an adhesive substrate to at least one formed blister prior to blister cleavage and harvesting the skin graft by removal of the adhesive substrate from blister cutting device following the blister cleavage.

12. The method of claim 1, wherein the blister cutting device comprises at least one fixed plate and at least one movable cutter plate, each plate having a plurality of holes through which blisters can be raised when holes of the fixed and movable plates are aligned, and the method further comprising cleaving the blisters by moving the movable cutter plate to disrupt alignment of the holes of the fixed and movable plates and thereby separate the blisters from remaining skin at the donor site.

13. The method of claim 1, wherein the device further comprises a plate with holes through which blisters can be raised and the method further comprises delivering a warm hypotonic fluid to a chamber within the device such that skin exposed to the chamber via the plate holes can assimilate fluid, at least one of before and after applying negative pressure to the chamber, to pull the skin into the chamber through the holes, and thereby raise a plurality of blisters.

14. A method for preparing a skin graft with a device comprising a device body, a sealing head member, at least one fixed plate and at least one movable cutter plate, each plate comprising a plurality of holes and wherein as assembled the holes in the plates are aligned within the body, the method comprising:
connecting the device to a donor site on a subject's skin;
joining the sealing head member and body together to define a sealed chamber;
applying negative pressure to the chamber to pull the subject's skin into the sealed chamber through the plurality of holes and thereby raise a plurality of blisters;
monitoring the formation of the plurality of blisters with an alternating current impedance sensor;
detecting when at least one blister is in a condition suitable for harvesting;
unsealing the chamber;
applying an adhesive substrate to blisters exposed within the chamber;
actuating the at least one movable cutter plate to disrupt alignment of holes in the at least one fixed plate and the at least one movable cutter plate and to cleave the blisters; and
removing the substrate together with the cleaved blisters.

15. The method of claim 14, further comprising applying heat to the donor site to assist in blister formation.

16. A device for obtaining a skin graft, the device comprising:
a cutting body configured to be disposable on a patient's skin;
a head configured to be coupled to the cutting body, the head further comprising a sealing surface configured to engage with a mating surface on the cutting body such that when the head is engaged with the body on the patient's skin, a sealed chamber is formed over a target region of skin;
a negative pressure conduit connected to the sealed chamber and configured to apply at least one of heat and a negative pressure within the sealed chamber to raise at least one skin blister within the sealed chamber;
an alternating current impedance sensor configured to monitor the formation of the at least one blister; and
a cutter assembly within the cutting body and configured to cleave the at least one blister after formation.

17. The device of claim 16, further comprising a heater configured to apply the heat within the sealed chamber to the target region of skin.

18. The device of claim 16, wherein the impedance sensor is configured to alert a user when the at least one blister has reached a state suitable for harvesting.

19. The device of claim 16, further comprising a controller configured to receive information from the impedance sensor, wherein the controller is further configured to at least one of a) initiate a reduction or a termination of the negative pressure based upon the information received from the sensor impedance and b) activate the cutter assembly to cleave the at least one blister based on information from the sensor.

20. The device of claim 19, wherein the controller and sensor are configured to communicate via a wireless communication protocol.

21. The device of claim 19, wherein the controller is configured to permit a user to select at least one parameter needed to initiate graft formation, the parameter being at least one of a desired blister height, a desired number of blisters, and a desired percentage of successfully formed blisters.

22. The device of claim 16, wherein the impedance sensor comprises at least one interrogating electrode and at least one receiving electrode within the device, wherein the sensor is configured to emit a signal from the interrogating electrode and receive the signal with the receiving electrode.

23. The device of claim 16, wherein the impedance sensor is disposed in close proximity to a growing blister and configured to sense at least one of formation and presence of a blister by detecting impedance.
